(19) United States
(12) Patent Application Publication
(10) Pub. No.: US 2003/0083635 A1
(43) Pub. Date: May 1, 2003

(54) Multidirectional side panels

(76) Inventor: Bernadette M. Gibbs, Statham, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 09/985,167

(22) Filed: Nov. 1, 2001

(51) Int. Cl. A61F 13/15; A61F 13/20

(52) U.S. Cl. 604/385.04; 604/385.24; 604/389; 604/391

(57) ABSTRACT

The present invention generally relates to an elastomeric side panel that is suitable for use in or on a disposable absorbent article. The elastomeric side panel is formed from an elastomeric material that has multidirectional stretch features enabling it to stretch in the longitudinal and lateral directions. The side panel has a first waist edge and a crotch edge that are substantially parallel to one another while in the relaxed condition. A fastening device is associated with the elastomeric side panel. A disposable absorbent article including the elastomeric side panel, a method of making the article, and a method of applying the article to a user, also are disclosed.

### FIELD OF THE INVENTION

The present invention generally relates to an elastomeric side panel (or “ear”) that is suitable for use in or on a disposable absorbent article. The elastomeric side panel is formed from an elastomeric material that has multidirectional stretch features enabling it to stretch in the longitudinal and lateral directions. A fastening device is associated with the elastomeric side panel. A disposable absorbent article including the elastomeric side panel also is disclosed.

### BACKGROUND OF THE INVENTION

Absorbent garments, such as diapers and adult incontinence products, often are constructed to have an hourglass or rectangular shape that is fitted between a wearer's legs, then wrapped around the wearer's waist, and then sealed on either side to form a pant-like structure on the wearer. Many of these garments have an elasticized waist that the caregiver stretches out while placing the garment on the wearer. The waist portion that typically is stretched is that portion normally associated with the back of a user. Thus, when the caregiver is placing the garment on the use, the back portion of the waist can be stretched around to the front waist portion and attached thereto to complete the waist of the garment.

The narrower portion of the known absorbent garments was adapted to be placed between the legs of the wearer with the wider portions of the article being adapted to encircle the waist of a wearer so that the front and rear portions overlapped and could be easily attached to one another. Recently, it has become desirable to produce absorbent articles, such as infant diapers, that fit more closely to the body of a wearer. Accordingly, it has become desirable to make such articles smaller and less conspicuous

In use while still maintaining a high level of absorbent protection. Specifically, it has become desirable to produce disposable absorbent articles which have a relatively narrow crotch section and a narrower overall width when compared to typical disposable absorbent articles.

Leg openings are defined, in part, on traditional infant diapers by the overlapped front and rear portions of the diaper. Such leg openings are generally perceived as providing good absorbent protection. If the front and rear portions of the diaper do not completely encircle the waist of a wearer and overlap with one another, there is a perception of decreased leakage performance. Known fastening devices for absorbent articles that do not completely encircle the waist of a wearer have generally consisted of relatively narrow rectangular devices or beltlike devices to hold the article in place around the waist of a wearer. Such known mechanisms do not generally correct for the perceived decrease in absorbent performance. Further, known fastening means for fastening the absorbent garments about the waist of a wearer can lead to undesirable red marking of the skin of a wearer if stretched too tightly. In addition, many of the fasteners may not attach the two halves of the garment to one another in an ideal manner, and may slip, loosen, or partially or completely open during use.

Introducing a degree of stretchability into personal care products such as diapers, sanitary napkins, adult incontinent products and the like is believed to provide these products with certain benefits. For example, U.S. Pat. No. 2,866,459 describes a stretchable, reusable cotton diaper. Stretchability is imparted to the cotton fabric by the presence of longitudinal and transverse elastic stitching. U.S. Pat. No. 3,371,668 discloses a sanitary napkin employing a nonwoven fabric with machine direction elasticity as a cover. The described fabric comprises a nonwoven web of fibers having a plurality of elastic means, in extended condition, secured to the web.

U.S. Pat. No. 4,606,964 describes a bulked web composite and method of making the same, where the composite is formed by adhering a reticulated web of elastic material to at least one gatherable web. Upon release of the tensioning forces, the gatherable web is gathered by the reticulated web to form a bulked web composite. U.S. Pat. No. 4,847,134 discloses a stretchable absorbent undergarment, including an inner layer or topsheet, a liquid impervious outer layer or backsheet, an absorbent layer and a stretchable layer. The stretchable layer comprises a continuous layer of stretchable material that may be perforated. These perforations are said to render the elastomeric layer permeable.

U.S. Pat. No. 5,957,908 discloses an elastomeric ear panel used with “convertible” absorbent articles, whereby the ear panels (or flaps) are said to enable the use of the article as a conventional diaper, or as a “pull-on” diaper. The specifically described ear panels impart the stretchability characteristics described therein. U.S. Pat. No. 5,938,652 discloses an absorbent article having a selectively elasticized waist flap that forms a pouch to contain the body exudates. The specific waist flap described therein is said to conform better to the wearer's back during use, while the remainder of the absorbent article is positioned away from the wearer's back, thus forming a pouch.

U.S. Pat. No. 5,451,219 discloses a stretchable absorbent article whereby the liquid permeable topsheet is connected to the liquid impermeable backsheet in discrete and non-discrete regions thereby forming a series of longitudinal pleats having channels disposed therein. The absorbent article is described as capable of absorbing a desirable amount of liquids, and at the same time capable of being elastically stretchable and contractible to better conform to the wearer's body during use. U.S. Pat. No. 5,411,498 discloses a stretchable shaped absorbent garment containing at least two generally parallel elastomeric strands stretch bonded to the garment along its length. When the article is in a relaxed state, the elastomeric strands are reduced in length rendering the article stretchable.

U.S. Pat. No. 5,496,298 discloses an elastomeric ear suitable for use on an absorbent garment. The elastomeric ear of this document has first and second connecting edges that are not parallel with one another to avoid undesirable red marking of the skin that occurs when the rear waist region of conventional absorbent garments are stretched and attached to the opposing waist region. The disclosures of all of the aforementioned United States patents are incorporated by reference herein in their entirety.

### SUMMARY OF THE INVENTION

It would be desirable to provide a garment that may be stretched without placing the fasteners under an undesirable tensile load. It would also be desirable to provide a garment that may be stretched to enable easy fastening, yet at the same time not utilize excess materials in the rear waist region. It also would be desirable to provide an absorbent garment that has a side panel and fastening mechanism that when fastened around the waist of the user, achieves a side panel contoured effect that shapes itself around the waist region of the user, and does not produce stress marks (typically indicated by red markings) on the skin of the user. It also would be desirable to provide a method of making a garment that has a side panel and fastening mechanism that provides the aforementioned desirable features. The present invention is designed to provide these desirable features.

It is therefore a feature of an embodiment of the invention to provide a garment the may be stretched and placed on an individual without placing the fasteners of the garment under an undesirable tensile load. It is an additional feature of an embodiment of the invention to provide a garment that stretches easily to enable easy fastening, but does not utilize excess materials in its manufacture. It is an additional feature of the invention to provide a garment having a contoured body fit when placed on a user even though the garment does not have a contoured configuration when in the unstretched condition. It is another feature of an embodiment of the invention to provide an absorbent garment that does not produce stress marks on the skin of the user when the garment is fitted around the waist of the user. It is an additional feature of an embodiment of the invention to provide an absorbent garment that is capable of fitting a wider variety of user sizes.

In accordance with these and other features of various embodiments of the invention, there is provided an absorbent garment having a topsheet, a backsheet, and an absorbent core disposed between the topsheet and the backsheet. The topsheet and the backsheet form a first waist region, a second waist region longitudinally opposite the first waist region, and a crotch region between the first and second waist regions.

The first waist region includes laterally extending side panels disposed on both lateral edges of the first waist region, where the laterally extending side panels each contain a fastening mechanism. The laterally extending side panels and the fastening mechanisms attached thereto have a first waist edge and a crotch edge, whereby the first waist edge and the crotch edge are substantially parallel to one another when the garment is in the relaxed, or unstretched, position. One or more target devices are attached to the garment in the second waist region, whereby the fastening mechanisms attached to the two side panels are capable of communicating or otherwise associating with the one or more target devices in the second waist region. The laterally extending side panels is made of a multidirectional elastomeric material. In another embodiment of the invention, at least one, and preferably both, the laterally extending side panels and the fastening mechanisms attached thereto are made of a multidirectional elastomeric material.

In accordance with another feature of an embodiment of the invention, there is provided a method of making an absorbent garment that includes preparing a topsheet, a backsheet, and an absorbent core, and disposing the absorbent core between the topsheet and the backsheet. The topsheet, backsheet, and absorbent core are prepared and arranged such that the topsheet and the backsheet form a first waist region, a second waist region longitudinally opposite the first waist region, and a crotch region between the waist regions. The method also includes preparing a pair of laterally extending side panels and attaching them to the first waist region in such a manner that they extend laterally away from opposite sides of the first waist region.

The method further includes preparing fastening mechanisms and attaching the fastening mechanisms to each laterally extending side panel, and preparing one or more target devices, and attaching the one or more target devices to the garment in the second waist region. In accordance with the method, the laterally extending side panels and the fastening mechanisms are prepared to have a first waist edge and a crotch edge, whereby the first waist edge and the crotch edge are substantially parallel to one another when the garment is in the relaxed, or unstretched, position.

In accordance with another feature of an embodiment of the invention, there is provided a method of applying a garment to a user including providing a garment that includes a topsheet, a backsheet, and an absorbent core disposed between the topsheet and the backsheet. The topsheet and the backsheet form a first waist region, a second waist region longitudinally opposite the first waist region, and a crotch region between the first and second waist regions. The first waist region includes laterally extending side panels disposed on both lateral edges of the first waist region, where the laterally extending side panels each contain a fastening mechanism. The laterally extending side panels have a first waist edge and a crotch edge, whereby the first waist edge and the crotch edge are substantially parallel to one another when the garment is in the relaxed, or unstretched, position. The garment also includes one or more target devices disposed on the second waist region.

The method includes placing the crotch region of the garment adjacent the crotch of a user such that either the first or second waist region is behind the user and the other waist region is in front of the user, both first and second waist regions being adjacent the waist of the user. The first waist region of the garment then is extended around the waist of the user, whereby the first waist region is extended such that at least its crotch edge is contoured, and is no longer substantially parallel to the first waist edge. The garment is finally fitted on the user by associating the fastening mechanism disposed on each side panel with the one or more target devices disposed on the second waist region.

These and other features of the invention will be readily apparent from the Detailed Description that follows, along with reference to the drawing appended hereto.

### BRIEF DESCRIPTION OF THE DRAWINGS

FIG. 1 is a partially cut-away view of an embodiment of the present invention, shown with elastic members fully stretched in the main portion of the garment, but with the elastic members unstretched in the laterally extending side panels;

FIG. 2 is a cross sectional view of the embodiment of FIG. 1, as seen from reference line AA, in an unstretched condition;

FIG. 3 is a plan view of one of the laterally extending side panels of FIG. 1 in the relaxed position;

FIG. 4 is a plan view of one of the laterally extending side panels of FIG. 1 in the stretched position;

FIG. 5 is a side view of the absorbent garment illustrated in FIG. 1 in use on a wearer;

FIG. 6 is a front view of the absorbent garment illustrated in FIG. 1 in use on a wearer;

FIG. 7 is a partially cut-away view of an alternative embodiment of the present invention, shown with elastic members fully stretched in the main portion of the garment, but with the elastic members unstretched in the laterally extending side panels and fastening members;

FIG. 8 is a cross sectional view of the embodiment of FIG. 7, as seen from reference line AA, in an unstretched condition;

FIG. 9 is a plan view of one of the laterally extending side panels and attached fastening mechanism of FIG. 7 in the relaxed position; and

FIG. 10 is a plan view of one of the laterally extending side panels and attached fastening mechanism of FIG. 7 in the stretched position.

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein.

Throughout this description, the expression “multidirectional elastic” denotes an elastic material that is capable of being stretched in the longitudinal and the lateral directions. Throughout this description, the expression “substantially parallel” denotes opposing sides that are essentially parallel with one another, whereby the angles between the two sides may vary by as much as 15 degrees. The relationship between the opposing sides preferably is determined when the component is fully relaxed, and the effects of the elastics removed (e.g., no shirring or gathering of the garment component is present). To the extent that the opposing sides are contoured sides, then the respective opposing contours are substantially parallel to one another, whereby the angles between the two contoured sides may vary by as much as 15 degrees.

The present invention relates to an absorbent garment, to a method of making the absorbent garment, and to a method of applying the absorbent garment to a user. The absorbent garment typically includes a topsheet, a backsheet, and an absorbent core disposed between the topsheet and backsheet. The topsheet and backsheet may be coterminous with one another, or one may be larger and “overlay” the other. The topsheet and the backsheet form a first waist region, a second waist region longitudinally opposite the first waist region, and a crotch region between the first and second waist regions.

The first waist region includes laterally extending side panels disposed on both lateral edges of the first waist region, where the laterally extending side panels each contain a fastening mechanism. The laterally extending side panels and the fastening mechanisms attached thereto have a first waist edge and a crotch edge, whereby the first waist edge and the crotch edge are substantially parallel to one another when the garment is in the relaxed, or unstretched, position. When the garment is stretched and subsequently fitted on a user, however, the first waist edge and the crotch edge are not substantially parallel to one another; rather, the crotch edge forms a contoured shape conforming itself around the user's legs. As an alternative embodiment, the crotch edge and first waist edge of the side panels are not substantially parallel to one another when fitted on a user, but the first waist edge and crotch edge of the fastening mechanisms are substantially parallel to one another.

One or more target devices are attached to the garment in the second waist region, whereby the fastening mechanisms attached to the two side panels are capable of communicating or otherwise associating with the one or more target devices in the second waist region. At least one of the laterally extending side panels and/or fastening mechanisms is made of a multidirectional elastomeric material. While not intending on being bound by any theory of operation, it is believed that the multidirectional elastomeric material enables the use of lesser amounts of material for the side panels such that, when in the relaxed position, the first waist edge and the crotch edge are substantially parallel, but when stretched, the multidirectional elastomeric material stretches laterally and longitudinally in such a contoured fashion that the first waist edge and the crotch edge are no longer substantially parallel.

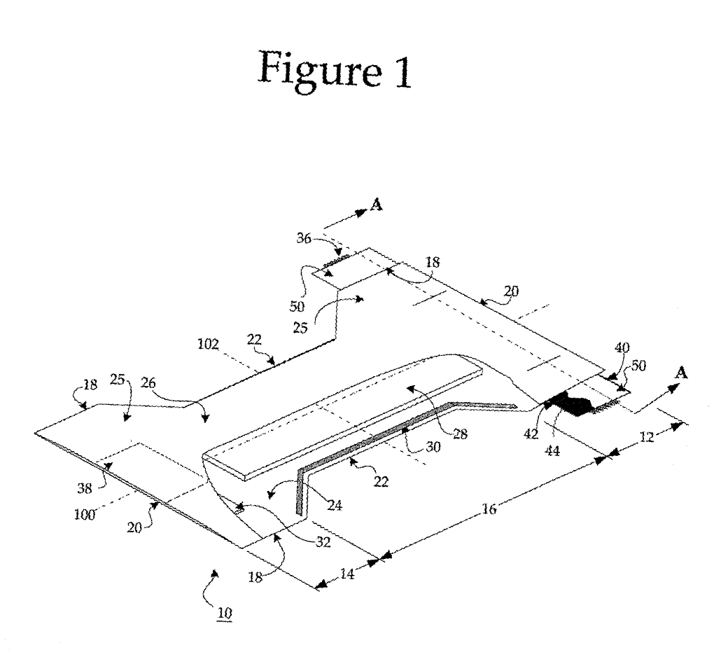

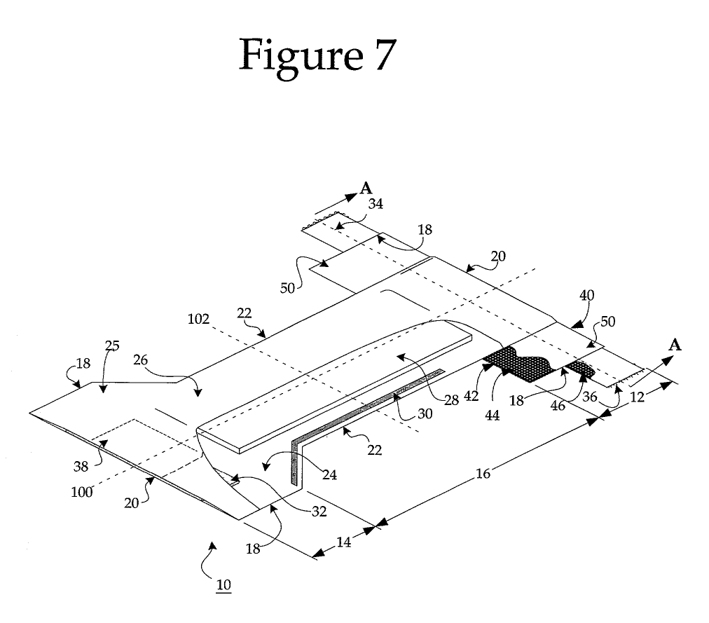

It is preferred in the present invention that the fastening mechanisms be placed directly on the side panel(s). An alternative embodiment of the invention, however, encompasses the use of separate fastening mechanisms, such as tabs and the like, that are attached or otherwise associated with the side panel(s). The preferred embodiments are depicted in FIGS. 1-4, and the alternative embodiments are depicted in FIGS. 7-10.

In one embodiment of the invention, the fastening mechanism may be a hook portion of a hook and loop fastener, and the one or more target devices may be the loop portion of a hook and loop fastener. In another embodiment, the fastener may be an adhesive tape and the target devices may comprise a tape receiving surface.

The invention now will be described with reference to the attached drawings illustrating preferred embodiments of the invention. For clarity, features that appear in more than one Figure have the same reference number in each Figure.

FIG. 1 is a partially cut away depiction of an exemplary embodiment of a garment 10 of the present invention. The embodiment shown in FIG. 1 is an infant's diaper, however, this depiction is not intended to limit the invention. The garment 10 of FIG. 1 is depicted in a generally flattened position, with the body-facing side facing down, and with the various elastic components depicted in their extended position for clarity. In the flattened position, the garment 10 may have a generally hourglass shaped structure, but it may also have any other shape suitable for the given application, such as a rectangular shape, a trapezoidal shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a topsheet 24, and a backsheet 26, which may be substantially coterminous with the topsheet 24. When the garment 10 is being worn, the topsheet 24 faces the wearer's body, and the backsheet 26 faces away from the wearer. An absorbent core 28 preferably is disposed between at least a portion of the topsheet 24 the backsheet 26.

An embodiment of the present invention may further comprise various additional features. One or more pairs of elastics gathers 30 may extend adjacent the crotch edges 22. The garment 10 may also comprise one or more waste containment systems, such as inboard standing leg gathers (not shown). One or both of the first and second waist regions 12, 14 may also be equipped with strips of elastic waist foam 32 or other elastically extensible material, which help contract the garment around the wearer's waist, providing improved fit and leakage prevention.

In the embodiment shown in both FIGS. 1 and 7, the garment 10 generally has an hourglass shape, with laterally extending wings 25. In the alternative embodiment shown in FIG. 7, however, the garment need not include wings 25 in the first waist region 12 where the side panels 50 and tabs 34 are disposed. Skilled artisans will appreciate that the alternative embodiment shown in FIG. 7 may include the same type of wings 25 as shown in FIG. 1, and that they may extend laterally to a greater or lesser extent, depending on the particular design.

In the alternative embodiment of FIG. 7, there is shown a pair of fastening mechanisms, including for example, tabs 34, extending laterally away from opposite lateral edges 18 of the first waist region 12 of the garment 10. The tabs 34 may comprise an elastically extensible material 46, preferably a multidirectional elastically extensible material 46, and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached either directly to the side panels in FIG. 1, or to each tab 34 in FIG. 7 for attaching the first waist region 12 to the second waist region 14, thereby providing the garment 10 with a pant-like shape, and enabling garment 10 to be fixed or otherwise fitted on the wearer. The fastening mechanisms 36 may attach to one or more target devices 38 located in the second waist region 14.

Although not shown in the drawings, the absorbent garment 10 may also include grips attached along one of its edges proximal to each tab 34 to enable a caregiver to pull the grips, and not on the ends of the tabs 34, around the wearer and over the target devices 38 to thereby secure the fasteners 36 to the one or more target devices 38.

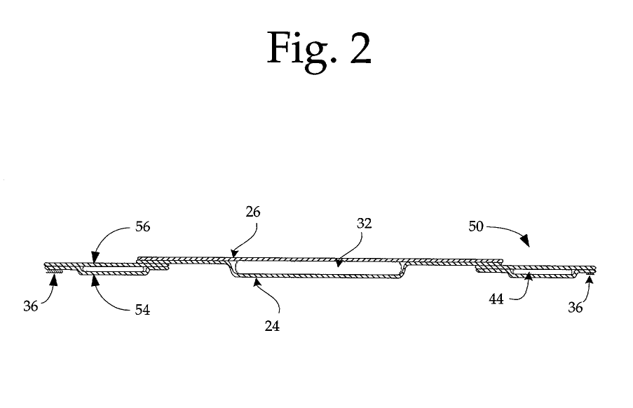

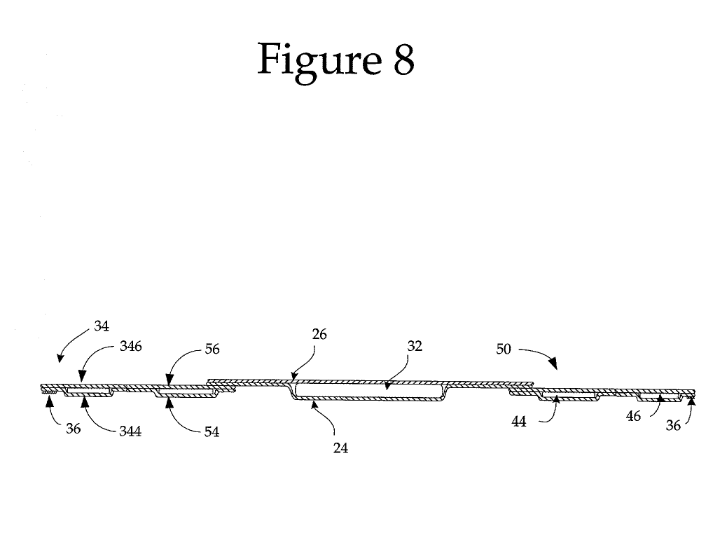

The garment 10 also includes a pair of laterally opposed side edges, or side panels 50. Side panels 50 include a first waist edge 40, and a longitudinally opposed crotch edge 42. Side panels 50 preferably are attached to the longitudinally extending lateral edges 18 of wings 25, as shown in FIG. 1. It also is preferred that side panel 50 be comprised of at least one multidirectional elastic material 44 disposed between upper 54 and lower 56 materials (FIGS. 2 and 8), which are used to make side panels 50. The lower material 56 may be made of the same or different material than the material used to make backsheet 26. Lower material 56 also may be treated in the same fashion so as to render it hydrophobic, and/or fluid impervious. Upper material 54 also may be comprised of the same or different material than the material used to make topsheet 24. Upper material 54 also may be treated in the same fashion so as to render it hydrophillic, and/or fluid pervious.

When garment 10 is in the unstretched or relaxed position, longitudinally opposing first waist edge 40 and crotch edge 42 of side panel 50 are substantially parallel to one another. It is preferred that the angle formed between first waist edge 40 and crotch edge 42 be less than 10 degrees, more preferably, less than 5 degrees, and most preferably less than 1 degree so that the respective edges are parallel.

In an alternative preferred embodiment of the invention, tabs 34 may each include a multidirectional elastic material 46 disposed between an upper material 344, and a lower material 346 (FIG. 8). The lower material 346 may be made of the same or different material than the material used to make backsheet 26. Lower material 346 also may be treated in the same fashion so as to render it hydrophobic, and/or fluid impervious. Upper material 344 also may be comprised of the same or different material than the material used to make topsheet 24. Upper material 344 also may be treated in the same fashion so as to render it hydrophillic, and/or fluid pervious.

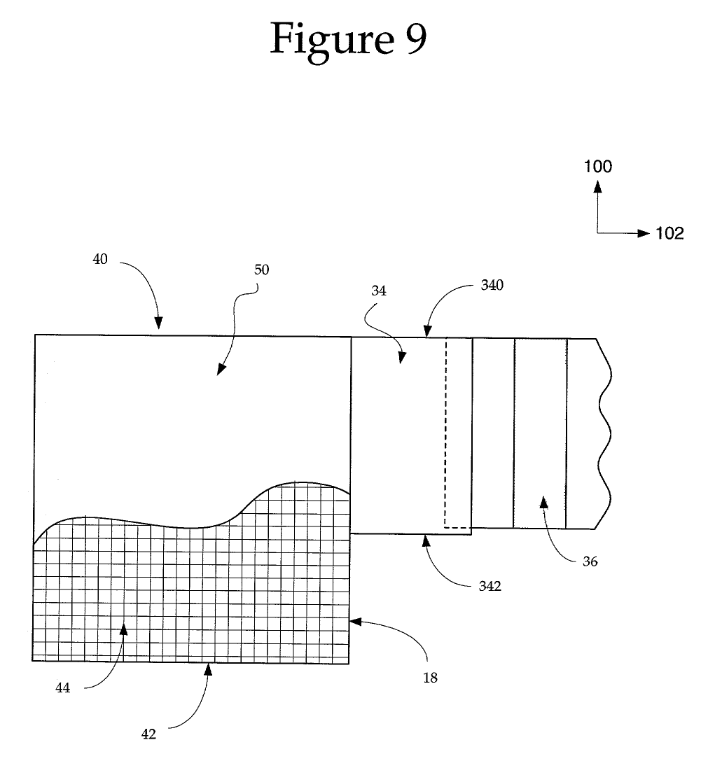

It also is preferred that tabs 34 have longitudinally opposed edges 340, 342, corresponding to a first waist edge 340, and a crotch edge 342 (FIG. 9). When garment 10 is in the unstretched or relaxed position, longitudinally opposing first waist edge 340 and crotch edge 342 of tabs 34 are substantially parallel to one another. It is preferred that the angle formed between first waist edge 340 and crotch edge 342 be less than 10 degrees, more preferably, less than 5 degrees, and most preferably less than 1 degree so that the respective edges are parallel.

In another embodiment of the invention, first waist edge 340 and crotch edge 342 of tabs 34 are substantially parallel to one another even after the tabs have been stretched and fixed to the one or more target devices 38 of garment 10. It is preferred that the angle formed between first waist edge 340 and crotch edge 342 after fixing tabs 34 to target devices 38 be less than 10 degrees, more preferably, less than 5 degrees, and most preferably less than 1 degree so that the respective edges are parallel. This embodiment of the invention encompasses those embodiments where multidirectional elastics 46 are present, where no elastic is present, or where a unidirectional elastic (not shown) material is present between upper material 344 and lower material 346. While not intending by being bound by any particular theory of operation, it is believed that the flexibility and stretchability of side panels 50 enables tabs 34 to retract to their original shape after stretching both tabs 34 and side panels 50 around the waist of a user. Accordingly, first waist edge 340 and crotch edge 342 of tabs 34 remain substantially parallel to one another even after fitting garment 10 on a wearer, but first waist edge 40 and crotch edge 42 of side panels 50 are not parallel after fitting garment 10 on a wearer.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The topsheet 24, backsheet 26, upper material 54 of side panel 50, upper material 344 of tab 34, lower material 56 of side panel 50, and lower material 346 of tab 34 may be constructed from a wide variety of materials known in the art. The invention is not intended to be limited to any specific materials for these components. The topsheet 24, backsheet 26, upper material 54 of side panel 50, upper material 344 of tab 34, lower material 56 of side panel 50, and lower material 346 of tab 34 can be shaped and sized according to the requirements of each of the various types of absorbent garment, or to accommodate various user sizes. In an embodiment of the invention in which the garment 10 is a diaper or an adult incontinence brief, the combination of topsheet 24, backsheet 26, upper material 54 of side panel 50, upper material 344 of tab 34, lower material 56 of side panel 50, and lower material 346 of tab 34 or any of these respective materials, may have an hourglass shape, as seen in FIG. 1, or may have a rectangular, trapezoidal, “T” shape, or other shape.

The backsheet 26, upper material 56 of side panel 50, and upper material 346 of tab 34 (hereinafter collectively referred to as “backsheet”) generally are made of any suitable pliable liquid impervious material known in the art or later discovered. Typical backsheet materials include films of polyethylene, polypropylene, polyester, nylon, and polyvinyl chloride and blends of these materials. For example, the backsheet 26 may be made of a polyethylene film having a thickness in the range of 0.02-0.04 mm. The backsheet 26 may be pigmented with, for example, titanium dioxide, to provide the garment 10 with a pleasing color or to render the backsheet 26 opaque enough that exudates being contained by the garment 10 are not visible from outside the garment. In addition, the backsheet 26 may be formed in such a manner that it is opaque, for example, by using various inert components in the polymeric film and then biaxially stretching the film. Other backsheet materials will be readily apparent to those skilled in the art. The backsheet 26 preferably should have sufficient liquid imperviousness to prevent any leakage of fluids through the backsheet 26. The required level of liquid imperviousness may vary between different locations on the garment 10.

The backsheet 26 may further comprise separate regions having different properties. In a preferred embodiment, portions of the backsheet 26 are air-permeable to improve the breathability, and therefore comfort, of the garment 10. The different regions may be formed by making the backsheet 26 a composite of different sheet materials, chemical treatment, heat treatment, or other processes or methods known in the art. Some regions of the backsheet 26 may be fluid pervious. In one embodiment of the invention, the backsheet 26 is fluid impervious in the crotch 16, but is fluid pervious in portions of the first and second waist regions 12, 14. The backsheet 26 may also be made from a laminate of overlaid sheets of material.

The backsheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with the present application and invention. Materials for such a fibrous outer liner include a spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers. Either the backsheet 26 or covering fabric may have imprinted thereon a characteristic mark, graphic, or illustration.

The topsheet 24, lower material 54 of side panel 50, and lower material 344 of tab 34 (hereinafter collectively referred to as “topsheet”) may be made of any suitable relatively liquid-pervious material currently known in the art or later discovered that permits passage of a liquid therethrough. Examples of suitable topsheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 28. The topsheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a topsheet material for the given application. The present invention is not intended to be limited to any particular material for the topsheet 24, and other topsheet materials will be readily apparent to those skilled in the art.

The topsheet 24 may further comprise several regions having different properties. In one embodiment of the present invention, the laterally distal portions of the topsheet 24, especially those used to make side panels 50 and tabs 34 (e.g., lower materials 54 and 344, respectively) are preferably substantially fluid impervious and hydrophobic, while the remainder of the topsheet 24 is hydrophilic and fluid pervious. Different topsheet properties, such as fluid perviousness and hydrophobicity, may be imparted upon the topsheet 24 by treating the topsheet 24 with adhesives, surfactants, or other chemicals, using a composite of different materials, or by other means. The topsheet 24 may also be made from a laminate of overlaid sheets of material. The topsheet 24 also may be treated in specific areas like the crotch region, with skin wellness ingredients such as aloe, vitamin E, and the like. Moreover, the topsheet 24 may be comprised of various regions where a central region is disposed adjacent the absorbent core 28 and is fluid pervious, and where outer regions include standing leg gathers (one or more than one pair) and are fluid impervious.

As noted elsewhere herein, the topsheet 24 and backsheet 26 may be substantially coterminous, or they may have different shapes and sizes. The particular design of the topsheet 24 and backsheet 26 may be dictated by manufacturing considerations, cost considerations, and performance considerations. Preferably, the topsheet 24 is large enough to completely cover the absorbent core 28, and the backsheet 26 is large enough to prevent leakage from the garment 10. The design of topsheet 24 and backsheet 26 is known in the art, and a skilled artisan will be able to produce an appropriate topsheet 24 and an appropriate backsheet 26 without undue experimentation.

The topsheet 24 and the backsheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the topsheet 24 to the backsheet 26. The particular joining method may be dictated by the types of materials selected for the topsheet 24 and backsheet 26.

An absorbent core 28 preferably is disposed between the topsheet 24 and the backsheet 26 in at least the crotch region 16. The absorbent core 28 may extend into either or both of the first and second waist regions 12, 14. Although the absorbent core 28 depicted in FIG. 1a has a substantially rectangular shape, other shapes may be used, such as a “T” shape or an hourglass shape. The shape of the absorbent core 28 may be selected to provide the greatest absorbency with a reduced amount of material. The absorbent core may be associated with the topsheet 24, backsheet 26, or any other suitable part of the garment 10 by any method known in the art, in order to fix the absorbent core 28 in place.

The absorbent core 28 may be made from any suitable material or materials known in the art that are capable of absorbing or retaining liquids (e.g., urine and/or menses). Examples of suitable materials for use as the absorbent core 28 include creped cellulose wadding, absorbent foams, absorbent sponges, super absorbent polymers, absorbent gelling materials, fiberized cellulose, chemically stiffened, modified or cross-linked cellulose fibers, fluff pulp having tissue or synthetic materials between the absorbent core 28 and the topsheet 24, comminuted wood pulp (commonly known as “airfelt”) or any equivalent material or combination of materials. The size and capacity of the absorbent material should correspond to the application, for example, an incontinent brief for an adult may require a larger absorbent core than a diaper for a child. Zoned absorbency may also be used, if desired. For example, more absorbent capacity may be located in particular regions of the garment 10 depending on the gender of the intended wearer. The invention is not intended to be limited to any specific materials or sizes for use in the absorbent core 28.

In a preferred embodiment, the absorbent core 28 comprises super absorbent polymer distributed within a fibrous structure. Absorbent cores of this type are known in the art, and exemplary absorbent cores are described in U.S. Pat. No. 5,281,207, issued to Chmielewski et al., and U.S. Pat. No. 5,863,288, issued to Baker, the disclosures of each of which are herein incorporated by reference in their entirety.

Additional sublayers, transfer layers, acquisition layers, tissue wraps, and the like also may be incorporated into the absorbent core 28. Such layers may be provided to assist with transferring fluids to the absorbent core 28, handling fluid surges, preventing rewet, containing absorbent material, improving core stability, or for other purposes. For example, a substantially rectangular, preferably nonwoven, sublayer (not shown), having a basis weight of about 0.1-2 oz., preferably about 0.4-0.6 oz., may overlay absorbent core 28. Those skilled in the art are capable of selecting materials, dimensions, and locations for such layers without undue experimentation.

In a preferred embodiment, the absorbent core 28 is a laminate comprised of a layer of superabsorbent polymer material in the form of particles disposed between two air-laid tissues, first and second tissue layers (or “upper” and “lower” tissue layers). The first and second tissue layers contain the superabsorbent polymer material, improve lateral wicking of the absorbed exudates throughout the absorbent core 28 and provide a degree of absorbency. Other absorbent laminate cores 28 can be used in the present invention, if desired.

The crotch region 16, or other regions of the garment 10, may also include mechanical sealing devices to provide the garment 10 with a leak-proof fit around the wearer. In a preferred embodiment, the crotch edges 22 each have one or more elastic leg gathers 30 placed along them to help the crotch edges 22 contract about the contours of the wearer's body, thereby providing a leak-resistant seal. Such elastic leg gathers 30 are known in the art, and are disclosed, for example, in U.S. Pat. No. 5,830,203, issued to Suzuki et al., the disclosure of which is herein incorporated by reference in its entirety in a manner consistent with the present invention. The elastic leg gathers 30 may extend into the first waist region 12, and may extend as far as the front longitudinally distal edge 20.

In another preferred embodiment, standing leg gathers (not shown) are disposed on the topsheet 24. Standing leg gathers are strips of material that rise vertically from the surface of the topsheet 24 to provide additional sealing to the garment 10. The standing leg gathers may extend across all or part of the garment 10 along its longitudinal axis 100. Typically, one or more standing leg gather is located laterally on either side of the absorbent core 28. The standing leg gathers may be made from folded portions of the topsheet 24 or backsheet 26, or may be made from additional strips of material. Each standing leg gather may be equipped with one or more elastic elements to help seal the gather to the wearer's body. Standing leg gathers are known in the art, and disclosed in U.S. Pat. No. 5,292,316, issued to Suzuki, the disclosure of which is herein incorporated by reference in its entirety in a manner consistent with the present invention.

Any suitable elastic material may be used for the elastic leg gathers 30 and the standing leg gathers. Preferably, the elastic material can be stretched to between at least 10% to 300% of its original length without losing its resilience. The elastic material used for the elastic leg gathers 30 and standing leg gathers may, for example, comprise an elastic film, a multidirectional elastic aggregate such as elastic webbing, netting, or scrim elastic, or strands or bands of suitable elastic materials, such as natural or synthetic rubber, urethane elastomers, spandex, LYCRA and elastic polymers. Other suitable elastics will be apparent to those skilled in the art in light of the present teachings. The elastics materials may be attached to the garment 10 in any of several ways known in the art. For example, the elastic materials may be ultrasonically bonded, heat/pressure sealed using a variety of bonding patterns, or glued to the diaper 10 using a variety of adhesives. Other performance enhancing devices, such as pockets, baffles, and openings in the topsheet also may be used with the present invention.

Any suitable multidirectional elastic material may be used as elastic material 44 or 46 in side panel 50 or tab 34, respectively. Multidirectional elastic materials are capable of being stretched in more than one direction. Particularly preferred multidirectional elastics include elastomeric materials, elastic webbing, netting, or scrim elastic, or strands or bands of suitable elastic materials that are disposed in the longitudinal and lateral directions, such as natural or synthetic rubber, urethane elastomers, spandex, LYCRA and elastic polymers. A particularly preferred multidirectional elastic material for use in the present invention is a scrim elastic material. Those skilled in the art are capable of providing a multidirectional elastic material 44 or 46 in side panel 50 or tab 34, respectively, and/or both, using the guidelines provided herein.

The first and second waist regions 12, 14 may be joined to one another by fastening mechanisms 36, or a plurality of fastening elements 36, each of which is attached directly to side panels 50, as shown in FIG. 1, or each of which is attached to a separate tab 34 protruding from an opposite lateral side edge 18 of the first waist region 12, as shown in FIG. 7. The fasteners 36 may interact with one or more target devices 38 located in the second waist region 14 to provide sufficient gripping force to hold the garment 10 on a wearer. While the embodiments depicted in FIG. 1 and show the tabs 34 and fastening mechanisms 36 being located in the first waist region 12, and the target device 38 being in the second waist region 14, skilled artisans will recognize that these locations may be reversed. Exemplary embodiments of the tabs 34, fasteners 36 and target devices 38 are provided herein, however, the present invention is not intended to be limited to any particular construction or materials for these parts.

The tabs 34, fasteners 36, and target devices 38, preferably are located and sized so that they may be used to wrap the first and second waist regions 12, 14 around wearers having a variety of sizes. For example, additional elastics (not shown) may be added to the garment 10 in one or both of the first and second waist regions 12, 14 in order to increase the range of user sizes that may be fitted by a single size garment 10. In a preferred embodiment, a strip of elastic foam 32 is located in each waist region to provide the waist regions with elasticity, additional comfort, and leakage prevention. The strips of elastic foam 32, such as an elastic polyurethane foam, also may help the longitudinally distal edges 20 to resist doubling over (“rolling”) during use.

The tabs 34 preferably are attached to the garment 10 at opposite lateral edges 18 of the first waist region 12, and extend distally away from the center of the garment 10. More specifically, tabs 34 are attached to the laterally distal edges 18 of side panels 50. The tabs 34 may be attached to the garment 10 using any method that provides a suitably strong bond. The tabs 34 may be attached in an edge-to-edge fashion with edges 18 of side panel 50, or the tabs 34 may overlap the topsheet 24, backsheet 26, upper material 54 of side panel 50, lower material 56 of side panel 50, or another part of the garment 10, in order to provide a stronger or larger bond area. An overlapping arrangement is depicted in FIG. 8, which is a cross sectional view of the embodiment of FIG. 7, as seen from reference line AA. Preferably, the tabs 34 are attached to one or both of the upper material 54 or lower material 56 of side panel 50. Exemplary methods for attaching the tabs 34 to the garment 10 include: ultrasonic welding, thermal bonding, adhesive bonding, stitching, stapling, combinations of the foregoing, and the like. A skilled artisan will be able to attach the tabs 34 to the garment 10 without undue experimentation.

The tabs 34 may comprise any suitable material. Nonwoven materials, such as those described elsewhere herein, may be particularly suitable for making the tabs 34. In a preferred embodiment, the tabs 34 comprise an elasticized material, such as a laminate of nonwoven sheets (e.g., upper material 344 and lower material 346), and an elastic material 46. Also in a preferred embodiment, the tabs are comprised of a material that is easily bonded to the garment 10 and to the fastening mechanisms 36 by any of various means known in the art.

The fastening mechanisms 36 may comprise an adhesive tape, a portion of a mechanically interlocking fastener, such as the hook or loop portion of a hook and loop fastener, or a button, snap, or any other suitable fastening mechanism known in the art. Preferably, the fastening mechanisms 36 may be sealed and released numerous times without losing their holding and/or fastening ability. The at least one target device 38 may be associated with a surface of the garment 10, or it may be an integral part of the surface of the garment. That is, the surface of the garment may itself act as a target device that will suitably interact with the fastening mechanisms 36. The selection and manufacture of fastening mechanisms 36, and target devices 38 is known in the art, and a skilled artisan will be able to implement such structures and devices without undue experimentation.

In a preferred embodiment, the fastening mechanism 36 is the hook portion of a hook and loop fastener. The fastening mechanism 36 may comprise a separate strip of material having a backing sheet with the hooks protruding therefrom, the fastening mechanism may be attached directly to side panel(s) 50, or the fastening mechanism 36 may protrude directly from the surface of each tab 34. The fastening mechanism 36 also may protrude beyond the laterally distal edges of the tabs 34. The design and construction of such tabs 34 and fastening mechanisms 36 are disclosed, for example, in U.S. Pat. No. 5,624,429 to Long et al., the disclosure of which is incorporated herein by reference in its entirety and in a manner consistent with the present invention.

Also in a preferred embodiment, the at least one target device 38 is the loop portion of a hook and loop fastener that is selected to mechanically engage with the fastening mechanism 36 to provide a secure, yet releasable and resealable bond. The target device 38 may be provided as a separate strip of material having a backing sheet with the loops protruding therefrom. In such a case, the target device or devices 38 may be attached to the outermost layer of the garment by any suitable means. In another embodiment, the target device 38 may also comprise a portion of the outer surface material of the garment (whether the outer surface is the backsheet 26 or an outer cover, as discusses elsewhere) that can mechanically engage with the fastening mechanisms 36, or that is mechanically or chemically treated to be able to mechanically engage with the fasteners 34.

In any case in which the fastening mechanisms 36 or target device 38 may cause irritation of the wearer's skin, it is preferred that such fastening mechanisms 36 or target devices 38 be shielded from contact with the wearer, such as by covering the target or fastening mechanism 38, 36 with a protective cover prior to being fastened together. Such a protective cover may also serve to prevent the target devices or fasteners from becoming contaminated and inoperable.

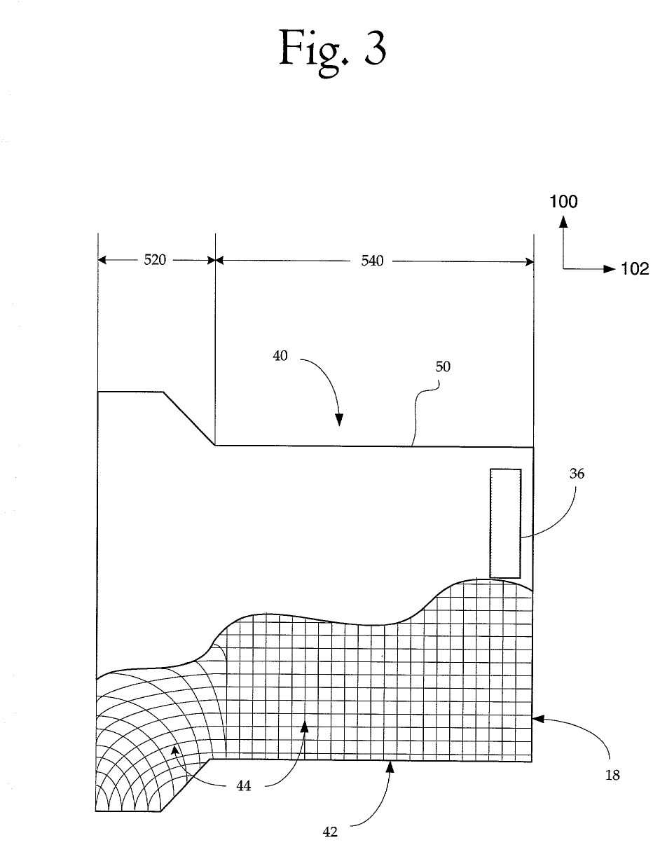

FIG. 2 is a cross sectional view of a portion of the embodiment shown in FIG. 1, as seen from reference line AA. FIGS. 2 and 3 also shows one of the many embodiments for attaching side panel(s) 50 to the main portion of the garment 10, preferably along the distal edges 18 of wings 25. An alternative embodiment shown in FIG. 8 is a cross sectional view of the embodiment shown in FIG. 7, as seen from reference line AA. FIG. 8 depicts another alternative method for attaching tab(s) 34 to side panel(s) 50. While this disclosure and reference to FIGS. 7-10 refers to side panel(s) 50 and tab(s) 34 being comprised of more than one item, those skilled in the art will appreciate that side panel(s) 50 and/or tab(s) 34 may be comprised of one unitary structure attached to first waist region 12 of garment 10, or two unitary structures comprised of side panel 50 and tab 34, each of which is attached to first waist region 12 of garment 10.

FIGS. 2 and 8 show elastic material 44 in side panel(s) 50, and elastic material 46 in tab(s) 34. Those skilled in the art will appreciate, however, that only side panel(s) 50 need contain the multidirectional elastic material, and that tab(s) 34 may contain a multidirectional elastic material 44, a unidirectional elastic material 44, no elastic material at all, or not exist at all as shown in FIG. 2. FIGS. 2 and 8 also depict the embodiment of the invention wherein waist foam element 32 is present in the first waist region 12, but skilled artisans will appreciate that first waist region 12 need not contain any elastic element, or it may contain elastic materials other than waist foam element 32.

As shown in FIG. 2, upper material 56 and lower material 54 of side panel 50 surround and otherwise envelop multidirectional elastic material 44. Upper material 56 and lower material 54 of side panel 50 may be made of the same material as backsheet 26 and topsheet 24, respectively, or they may be made of other materials, as described above. Other layers also may be present between upper material 56 and multidirectional elastic 44, and/or lower material 54 and multidirectional elastic 44. In addition, other layers may be disposed outside upper material 56 and/or lower material 54 of side panel 50.

Skilled artisans will appreciate that fastening mechanisms 36 attached to side panels 50 may be disposed on the lateral edge of the side panels, or they may be attached anywhere along side panel 50. For example, fastening mechanisms 36 may be disposed near the middle of side panels 50 (longitudinal as well as lateral) so that the pulling tension on the fastening mechanism can be altered. Those skilled in the art are capable of designing any suitable side panel 50 and fastening mechanism 36 arrangement, using the guidelines provided herein.

As an alternative embodiment shown in FIG. 8, upper material 346 and lower material 344 of tab 34 surround and otherwise envelop multidirectional elastic material 46. Upper material 346 and lower material 344 of tab 34 may be made of the same material as backsheet 26 and topsheet 24, respectively, or they may be made of other materials, as described above. While the embodiment shown in FIG. 8 illustrates elastic material 46 present in tab 34, no elastic material need be present, or the elastic material need not be a multidirectional elastic. Other layers also may be present between upper material 346 and multidirectional elastic 46, and/or lower material 344 and multidirectional elastic 46. In addition, other layers may be disposed outside upper material 346 and/or lower material 344 of tab 34.

FIG. 3 is a plan view of side panel 50 as it is preferably attached to garment 10 at or near the distal edges 18 of wings 25. It is preferred that side panels be stretched longitudinally prior to attaching to garment 10. As mentioned previously, side panels 50 may be attached to garment 10 either by overlapping and bonding respective parts together, as shown in FIG. 2, or by bonding along the distal edges of the respective component parts. In FIG. 3, it can be seen that stretching panels 50 prior to attachment to wings 25 provides a panel having essentially two portions; an attached portion 520 and an unattached portion 540. Due to stretching, the multidirectional elastic members 44 in attached portion 520 will be partially displaced, as depicted in FIG. 3. In contrast, the multidirectional elastic members 44 in unattached portion 540 will not be displaced. Consequently, first waist edge 40 and crotch edge 42 of side panel will be substantially parallel to one another, when in the relaxed, unstretched condition.

It is preferred to attach side panels 50 to garment 10 in this manner to reduce the stress and tension generated when the garment 10 is finally fitted on a user. In the preferred embodiment depicted in FIG. 3, the attached portion 520 of side panels 50 already are under tension prior to bonding or otherwise associating them with garment 10. It is therefore believed that less tension will be generated at the bonding points between attached portion 520 and garment 10 when the side panels 50 are stretched and fitted on a user. As a consequence, side panels 50 in this configuration are less likely to be pulled off of the garment during stretching and use. It also is preferred in this embodiment of the invention that fastening mechanisms 36 be attached directly to the unattached portion 540 of side panel 50.

FIG. 9 is a plan view of side panel 50 and tab 34 in the relaxed state. The particular proportions illustrated in the attached figures are not intended to be limiting of the present invention. Side panel 50 can be made to be any size, depending primarily on the size of the intended wearer, and hence on the size of the main portion of the absorbent garment (e.g., that comprised of topsheet 24 and backsheet 26). An advantage of the present invention, however, is that less material can be used for side panel 50, when compared to conventional absorbent garments that use side panels having contoured edges, and at least a contoured crotch edge. For example, the side panels 50 of the present invention that include multidirectional elastics can use 15% less material than contoured crotch edge side panels used in a similarly sized absorbent garment, preferably, 25% less material, more preferably, 20% less material, and most preferably, 50% less material.

It is preferred in the invention that side panels 50, whether those used in the preferred embodiment show in FIGS. 1-4, or an alternative embodiment shown in FIGS. 7-10, have an area anywhere from about a 400 mm² to about 10,000 mm², preferably from about 900 mm² to about 5,000 mm², and most preferably from about 2,000 to about 3,600 mm². It is most preferred in the invention that side panels 50 be in the shape of a square, although other shapes are possible in the invention, and even most preferably, a square of about 50 by 50 mm.

FIG. 9 depicts tab 34 and side panel 50 as separate materials, but those skilled in the art recognize that they may be comprised of one unitary article. FIG. 3 also shows first waist edge 40 and crotch edge 42 of side panel 50 as being substantially parallel to one another while in the relaxed state. In a similar fashion, first waist edge 340 and crotch edge 342 are substantially parallel to one another while in the relaxed state. Side panel 50 also is shown to include multidirectional elastics 44. Tab 34 also may include multidirectional elastics 46, other types of elastics 46, or no elastic material at all. In addition, the relationship between tab 34 and side panel 50 is illustrated in FIG. 9 as essentially being coterminous at the first waist edges 40 and 340. That is, first waist edge 40 of side panel 50, and first waist edge 340 of tab 34 form essentially a single line or curve. This configuration also is not intended to be limiting of the invention, and tab 34 and side panel 50 may be related to one another in any of the many well known configurations.

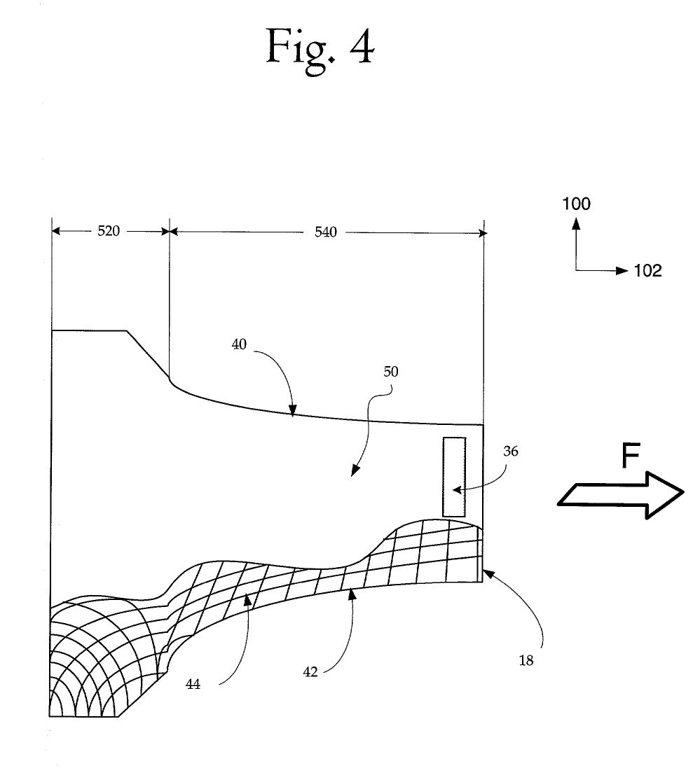

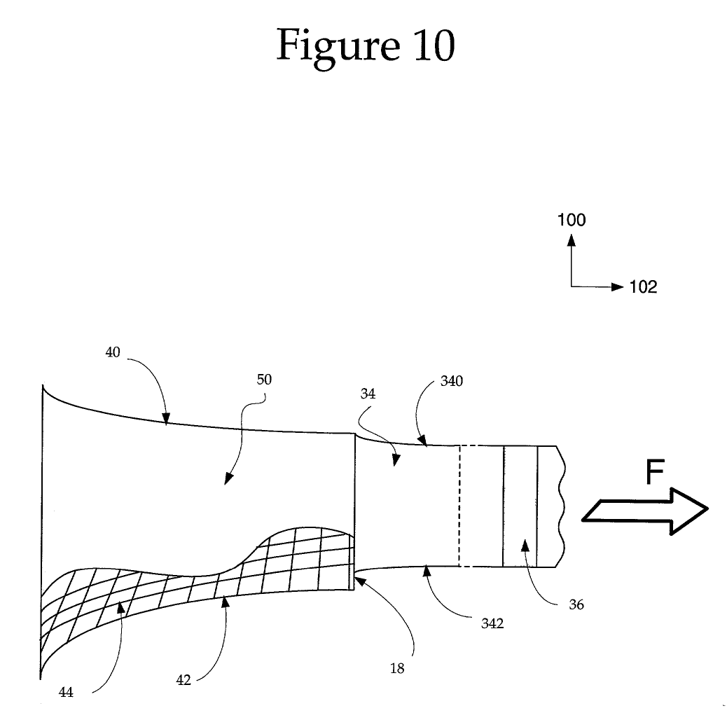

FIGS. 4 and 10 are plan views of the side panel 50 and tab 34 of FIGS. 3 and 9 in the stretched state after a force F has been applied in the lateral direction (102 in FIG. 4). Due to the presence of multidirectional elastics 44 in side panel 50, crotch edge 42 no longer is substantially parallel to first waist edge 40. No longer substantially parallel in this context denotes the respective edges being at an angle of greater than about 5 degrees, preferably greater than about 10 degrees, and most preferably greater than about 15 degrees from one another. The angle between the respective edges of contoured edges, such as those shown in FIGS. 4 and 10, can be determined by drawing a tangent at the greatest degree of curvature of the contoured edges, extending the respective tangents until they intersect with one another, and then measuring the angle formed by the intersecting tangent lines.

FIG. 10 also shows tab 34 as having contoured first waist edge 340 and crotch edge 342, upon application of force F in the lateral direction. This embodiment therefore includes multidirectional elastics 46 in tab 34, but again, skilled artisans recognize that such elastics need not be present. In addition, those skilled in the art will recognize that even if multidirectional elastics 46, or other types of elastics, are present in tab 34, the fastening mechanisms 36 may be attached to the at least one target devices 38 while in the stretched condition, and that when contracted, side panels 50 may bear the brunt of the stretching force. In this case, the tabs 34 may not be stretched, and accordingly, first waist edge 340 and crotch edge 342 of tab 34 may be substantially parallel to one another even after attachment to at least one target devices 38.

As in FIGS. 3 and 9, the particular proportions illustrated in the FIGS. 4 and 10 are not intended to be limiting of the present invention. Side panel 50 can be made to be any size, depending primarily on the size of the intended wearer, and hence on the size of the main portion of the absorbent garment (e.g., that comprised of topsheet 24 and backsheet 26, as well as the size of wings 25). In addition, the relationship between tab 34 and side panel 50 is illustrated in FIG. 10 as essentially being coterminous at the first waist edges 40 and 340. That is, first waist edge 40 of side panel 50, and first waist edge 340 of tab 34 form essentially a single line or curve. This configuration also is not intended to be limiting of the invention, and tab 34 and side panel 50 may be related to one another in any of the many well known configurations. For example, crotch edge 42 of side panel 50 and crotch edge 342 of tab 34 may be coterminous, or neither longitudinal edges of side panel 50 and tab 34 are coterminous.

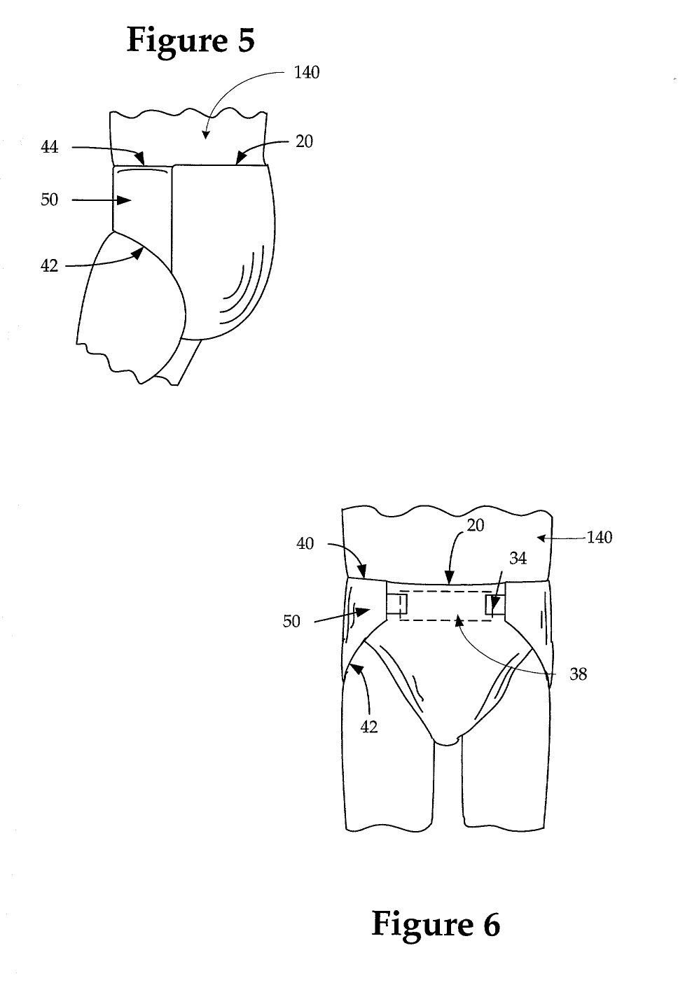

FIGS. 5 and 6 illustrate a preferred absorbent garment 10 as it is fitted in on a wearer. It is particularly preferred in the invention that the garment 10 is not a pull-on-pants-type garment, as typically used for training pants. These particular garments typically have extensive elastic elements disposed in the hip regions to allow for improved stretchability. Rather, it is preferred that garment 10 be a disposable diaper or incontinence garment that can be attached and separated repeatedly from the user by virtue of fastening mechanisms 36 and the at least one target device 38.

As shown, crotch edge 42 of side panel 50 is contoured after the garment is stretched and fitted on a wearer. In addition, first waist edge 40 and crotch edge 42 of side panel 50 are not substantially parallel to one another when the garment is fitted on a wearer. FIG. 6 shows an alternative embodiment of the invention where the first waist edge 340 and crotch edge 342 of tab 34 are substantially parallel to one another, even after fastening mechanisms 36 have been attached to the target devices 38.

Another alternative embodiment of the invention is one in which the side panels 50 comprise multidirectional elastic materials, and function essentially like conventional tabs. In this alternative embodiment, the first waist edge 40 and crotch edge 42 may remain substantially parallel to one another even after stretching and fitting the garment 10 on a user. In this and other embodiments of the invention, it is preferred that first waist edge 40 of side panels 50 not be coterminous with first waist edge 20 of garment 10. Rather, first waist edge 40 of side panels 50 preferably are placed at a position closer to the crotch region than longitudinally distal first waist edge 20.

In another embodiment of the invention, there is provided a method of making an absorbent garment that includes preparing a topsheet 24, a backsheet 26, and an absorbent core 28, and disposing the absorbent core between the topsheet 24 and the backsheet 26. The topsheet 24, backsheet 26, and absorbent core 28 are prepared and arranged such that the topsheet 24 and the backsheet 26 form a first waist region 12, a second waist region 14 longitudinally opposite the first waist region 12, and a crotch region 16 extending between the waist regions 12, 14. The method also includes preparing a pair of laterally extending side panels 50 and attaching them to the first waist region 12 in such a manner that they extend laterally away from opposite sides 18 of the first waist region 12.

The method further includes preparing fastening mechanisms 36 and attaching the fastening mechanisms 36 to each laterally extending side panel 50, and preparing one or more target devices 38, and attaching the one or more target devices to the garment in the second waist region 14. In accordance with the method, the laterally extending side panels 50 and the fastening mechanisms 36 are prepared to have a first waist edge 40 and a crotch edge 42, whereby the first waist edge 40 and the crotch edge 42 are substantially parallel to one another when the garment is in the relaxed, or unstretched, position.

In the method, it is preferred that the laterally extending side panels 50 are stretched in the longitudinal direction 100 prior to attaching to wings 25 of garment 10. Thus, as shown in FIG. 3, the attached portion 520 of side panels 50 will be extended longitudinally to a greater extent than unextended portion 540 of side panels 50, and the multidirectional elastic material 44 in the attached portion 520 will be displaced, when compared to the multidirectional elastic material 44 in the relaxed, unstreteched position in unattached portion 540.

Side panels 50 may either comprise fastening elements 36 and therefore function similarly to laterally extending tabs, or additional laterally extending tabs 34 may be disposed at the laterally distal edges 18 of side panels 50, as shown in FIG. 7. Skilled artisans are capable of manufacturing garment 10 in either of these embodiments, using the guidelines provided herein.

Any mechanism used to form the absorbent garment of the invention can be used herein. For example, multiple rollers, feed rollers, nip rollers, heated rollers, ultrasonic horns and anvils, can be used to provide the respective materials to a forming station where they are brought into communication with one another. The portions of the garment then can be attached or associated with one another at the forming station, cut into the appropriate size and shape, printed, or otherwise further processed, as is well known in the art. Any of the apparatus known and conventional in the art can be used to make the absorbent garment of the present invention, and those skilled in the art are capable of designing a suitable apparatus to make the absorbent garment described herein, using the guidelines provided herein.

In another embodiment of the invention, there is provided a method of applying a garment to a user including providing a garment 10 that includes a topsheet 24, a backsheet 26, and an absorbent core 28 disposed between the topsheet 224 and the backsheet 26. The topsheet 24 and the backsheet 26 form a first waist region 12, a second waist region 14 longitudinally opposite the first waist region 12, and a crotch region 16 extending between the first and second waist regions 12, 14. The first waist region 12 includes laterally extending side panels 50 disposed on both lateral edges of the first waist region, where the laterally extending side panels 50 each contain a fastening mechanism 36. The laterally extending side panels 50 have a first waist edge 40 and a crotch edge 42, whereby the first waist edge 40 and the crotch edge 42 are substantially parallel to one another when the garment 10 is in the relaxed, or unstretched, position. The garment also includes one or more target devices 38 disposed on the second waist region 14.

The method includes placing the crotch region 16 of the garment adjacent the crotch of a user such that either the first or second waist region 12, 14 is behind the user and the other waist region is in front of the user, both first and second waist regions 12, 14 being adjacent the waist of the user 140 (see, FIGS. 5 and 6). The first waist region 12 of the garment then is extended around the waist 140 of the user, whereby the side panels 50 are extended such that at least their crotch edges are contoured, and no longer substantially parallel to the first waist edge 40. The garment is finally fitted on the user by associating the fastening mechanism 36 disposed on each side panel 50 with the one or more target devices 38 disposed on the second waist region 14.

An alternative method involves placing the garment depicted in FIGS. 7-10 on a user. In this embodiment, both the laterally extending tabs 34 and side panels 50 may include multidirectional elastics 46,44, respectively. In this embodiment the first waist edge 340 and crotch edge 342 of tab 34 may remain substantially parallel to one another after attaching the garment 10 to the user. In addition, in any of the embodiments of the invention, the first waist edge 40 and crotch edge 42 of side panels 50 may remain substantially parallel to one another after attaching the garment 10 to the user.

The invention now will be explained with reference to the following examples.

### EXAMPLES

Three prototype absorbent garments in the form of a disposable diaper were prepared by attaching three side panels 50 to wings 25 of an absorbent garment in a manner as shown in FIG. 1. The side panels ranged from about a 40 mm×50 mm material, to a 60×50 mm side panel.

One of the side panels was comprised of a 50 mm×50 mm material. Fastening mechanisms 36, in the form of hooks, were attached to the side panels 50. The fastening mechanisms were disposed in the lateral distal portions of side panels 50. One of the fastening elements extended longitudinally across the entire distal end of side panel 50, another of the fastening elements was disposed in the center portion of the laterally distal edges, and a third fastening element extended a longitudinal distance greater than the longitudinal width of side panel 50. All side panels were attached to wings 25 in the manner shown in FIG. 3 by first stretching the attached portion 520, and then attaching it to the wings 25.

The side panels 50 for all three prototypes were comprised of two outer sheets of non-woven material surrounding a scrim multidirectional elastic material. When in the fully relaxed position, and unshirred, the first waist edge 40 and the crotch edge 42 are substantially parallel to one another. When stretched and placed around the waist of a simulated baby dummy, the diaper formed a contoured edge about the legs, as shown in FIGS. 5 and 6.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent garment having a longitudinal dimension and a lateral dimension comprising:
a topsheet;
a backsheet, whereby the topsheet and the backsheet form a first waist region, a second waist region longitudinally opposite the first waist region, and a crotch region therebetween;
an absorbent core disposed between the topsheet and the backsheet;
laterally extending side panels comprised of a multidirectional elastomeric material, the side panels being disposed on both lateral edges of the first waist region, whereby the laterally extending side panels include a first waist edge and a crotch edge;
at least one fastening mechanism attached to the each side panel; and
one or more target devices attached to the garment in the second waist region, where at least one fastening element and the one or more target devices are capable of attaching to one another, the one or more target devices being located so that the first waist region and second waist region of the garment may be joined to one another to secure the garment on a wearer;
whereby the first waist edge and the crotch edge of the laterally extending side panels are substantially parallel to one another when the garment is in the relaxed position.

2. The absorbent garment of claim 1, wherein each side panel comprises a portion of at least one of the topsheet and backsheet.

3. The absorbent garment of claim 1, wherein the laterally extending side panels comprise separate pieces of material attached to the garment at or near opposite lateral wings of the first waist region.

4. The absorbent garment of claim 1, wherein the at least one fastening element comprises a hook portion of a hook and loop fastener and the one or more target devices comprise the loop portion of a hook and loop fastener.

5. The absorbent garment of claim 1, wherein the at least one fastening element is an adhesive tape and the one or more target devices comprise a tape receiving surface.

6. The absorbent garment of claim 1, wherein the multidirectional elastic is selected from the group consisting of elastomeric materials, elastic webbing, netting, scrim elastic, and strands or bands of elastic materials that are disposed in the longitudinal and lateral directions

7. The absorbent garment of claim 6, wherein the strands or bands of elastic materials are selected from the group consisting of natural or synthetic rubber, urethane elastomers, spandex, LYCRA, elastic polymers, and mixtures thereof.

8. The absorbent garment of claim 6, wherein the multidirectional elastic material is scrim elastic.

9. The absorbent garment of claim 1, wherein the side panels are comprised of two separate panels.

10. The absorbent garment of claim 1, wherein the side panels are comprised of a unitary article.

11. The absorbent garment as claimed in claim 1, wherein the side panels are comprised of an upper material, a lower material, and the multidirectional elastic, whereby the multidirectional elastic is disposed between the upper material and the lower material.

12. The absorbent garment as claimed in claim 11, wherein the upper material and the lower material are comprised of the same material as the backsheet.

13. The absorbent garment of claim 1, wherein the at least one fastening mechanism is comprised of a pair of laterally extending tabs disposed on the lateral edges of the side panels, whereby the laterally extending tabs each include at least one fastening mechanism.

14. The absorbent garment of claim 13, wherein the laterally extending tabs comprise an elastic material.

15. The absorbent garment of claim 14, wherein the elastic material is a multidirectional elastic material.

16. The absorbent garment of claim 13, wherein the laterally extending tabs are comprised of an upper material, a lower material, and the elastic material, whereby the elastic material is disposed between the upper material and the lower material.

17. The absorbent garment as claimed in claim 16, wherein the upper material and the lower material are comprised of the same material as the backsheet.

18. The absorbent garment as claimed in 13, wherein the tabs are comprised of two separate elements each attached to the lateral edges of the side panels.

19. The absorbent garment of claim 13, wherein the tabs are comprised of a unitary article.

20. The absorbent garment as claimed in claim 13, wherein the side panels and the at least one fastening element are comprised of a unitary article.

21. The absorbent garment as claimed in claim 1, wherein the first waist edge and crotch edge of the side panels are not substantially parallel when the garment is placed on a wearer.

22. A method of making an absorbent article comprising:
preparing a topsheet, a backsheet, and an absorbent core;
disposing the absorbent core between the topsheet and the backsheet, whereby the topsheet, backsheet, and absorbent core are prepared and arranged such that the topsheet and the backsheet form a first waist region, a second waist region longitudinally opposite the first waist region, and a crotch region between the waist regions;
preparing a pair of laterally extending side panels;
attaching the laterally extending side panels to the first waist region in such a manner that they extend laterally away from opposite sides of the first waist region;
preparing fastening mechanisms and attaching the fastening mechanisms to each laterally extending side panel; and
preparing one or more target devices, and attaching the one or more target devices to the garment in the second waist region, whereby the laterally extending side panels have a first waist edge and a crotch edge, whereby the first waist edge and the crotch edge are substantially parallel to one another when the garment is in the relaxed position.

23. The method of claim 22, wherein each side panel comprises a portion of at least one of the topsheet and backsheet.

24. The method of claim 22, wherein the laterally extending side panels comprise separate pieces of material attached to the garment at or near opposite lateral wings of the first waist region.

25. The method of claim 22, wherein the at least one fastening element comprises a hook portion of a hook and loop fastener and the one or more target devices comprise the loop portion of a hook and loop fastener.

26. The method of claim 22, wherein the at least one fastening element is an adhesive tape and the one or more target devices comprise a tape receiving surface.

27. The method of claim 22, wherein the multidirectional elastic is selected from the group consisting of elastomeric material elastic webbing, netting, scrim elastic, and strands or bands of elastic materials that are disposed in the longitudinal and lateral directions

28. The method of claim 27, wherein the strands or bands of elastic materials are selected from the group consisting of natural or synthetic rubber, urethane elastomers, spandex, LYCRA, elastic polymers, and mixtures thereof.

29. The method of claim 27, wherein the multidirectional elastic material is scrim elastic.

30. The method of claim 22, wherein the side panels are comprised of two separate panels.

31. The method of claim 22, wherein the side panels are comprised of a unitary article.

32. The method as claimed in claim 22, wherein preparing the side panels comprises providing an upper material, a lower material, and a multidirectional elastic, and disposing the multidirectional elastic between the upper material and the lower material.

33. The method as claimed in claim 32, wherein the upper material and the lower material are comprised of the same material as the backsheet.

34. The method of claim 22, wherein the at least one fastening mechanism is comprised of a pair of laterally extending tabs, attaching the laterally extending tabs to the side panel comprises attaching the tabs to the lateral edges of the side panels, and whereby the laterally extending tabs each include at least one fastening mechanism.

35. The method of claim 34, wherein the laterally extending tabs comprise an elastic material.

36. The method of claim 35, wherein the elastic material is a multidirectional elastic material.

37. The method of claim 34, wherein the laterally extending tabs are prepared by providing an upper material, a lower material, and an elastic material, and disposing the elastic material between the upper material and the lower material.

38. The method as claimed in claim 37, wherein the upper material and the lower material are comprised of the same material as the backsheet.

39. The method as claimed in 34, wherein the tabs are comprised of two separate elements each attached to the lateral edges of the side panels.

40. The method of claim 34, wherein the tabs are comprised of a unitary article.

41. The method as claimed in claim 34, wherein the side panels and the at least one fastening element are comprised of a unitary article.

42. The method as claimed in claim 22, wherein the first waist edge and crotch edge of the side panels are not substantially parallel when the garment is placed on a wearer.

43. The absorbent garment as claimed in claim 1, wherein the first waist edge and crotch edge of the side panels are substantially parallel when the garment is placed on a wearer.

44. The method as claimed in claim 22, wherein the first waist edge and crotch edge of the side panels are substantially parallel when the garment is placed on a wearer.

45. A method of applying a garment to a user, the garment having a longitudinal dimension and a lateral dimension and comprising:
a topsheet;
a backsheet, whereby the topsheet and the backsheet form a first waist region, a second waist region longitudinally opposite the first waist region, and a crotch region there between;
an absorbent core disposed between the topsheet and the backsheet;
laterally extending side panels comprised of a multidirectional elastomeric material, the side panels being disposed on both lateral edges of the first waist region, whereby the laterally extending side panels include a first waist edge and a crotch edge;
at least one fastening mechanism attached to the each side panel; and
one or more target devices attached to the garment in the second waist region;
whereby the first waist edge and the crotch edge of the laterally extending side panels are substantially parallel to one another when the garment is in the relaxed position; the method comprising:
placing the crotch region of the garment adjacent the crotch of a user such that either the first or second waist region is behind the user and the other waist region is in front of the user, both first and second waist regions being adjacent the waist of the user;
extending the first waist region of the garment around the waist of the user; and
fitting the extended garment on the user by associating the fastening mechanism disposed on each side panel with the one or more target devices disposed on the second waist region.

46. The method as claimed in claim 45, wherein the first waist region is extended such that at least its crotch edge is contoured, and is no longer substantially parallel to the first waist edge.

47. The method as claimed in claim 45, wherein the first waist region is extended such that at least its crotch edge is substantially parallel to the first waist edge.

48. The method as claimed in claim 45, wherein the side panels are comprised of an upper material, a lower material, and the multidirectional elastic, whereby the multidirectional elastic is disposed between the upper material and the lower material.

49. The method as claimed in claim 48, wherein the upper material and the lower material are comprised of the same material as the backsheet.

50. The method of claim 48, wherein the at least one fastening mechanism is comprised of a pair of laterally extending tabs disposed on the lateral edges of the side panels, whereby the laterally extending tabs each include at least one fastening mechanism.

51. The method of claim 50, wherein the laterally extending tabs comprise an elastic material.

52. The method of claim 51, wherein the elastic material is a multidirectional elastic material.

53. The method of claim 50, wherein the laterally extending tabs are comprised of an upper material, a lower material, and the elastic material, whereby the elastic material is disposed between the upper material and the lower material.

54. The method as claimed in claim 53, wherein the upper material and the lower material are comprised of the same material as the backsheet.
